(19) United States
(12) Patent Application Publication
(10) Pub. No.: US 2026/0210567 A1
(43) Pub. Date: Jul. 23, 2026

(54) SCENT DELIVERY SYSTEM AND REGISTER ASSEMBLY WITH INTEGRATED SCENT CARTRIDGE RECEPTACLE

(71) Applicant: Scent A Filter LLC, Carrollton, TX (US)

(72) Inventor: Jeremy Van Winkle, Frisco, TX (US)

(21) Appl. No.: 19/450,928

(22) Filed: Jan. 16, 2026

Related U.S. Application Data
(60) Provisional application No. 63/746,710, filed on Jan. 17, 2025.

(51) Int. Cl. F24F 8/50; A61L 9/12; B01D 46/00; F24F 13/28

(52) U.S. Cl. CPC F24F 8/50; A61L 9/12; B01D 46/0038; F24F 13/28; A61L 2209/133; A61L 2209/14; A61L 2209/15; A61L 2209/16; B01D 2279/50

(57) ABSTRACT

A scent delivery system for use with an HVAC filter includes a housing member comprising a body and a sidewall extending from the body, the sidewall defining a cavity. A substrate member disposed within the cavity retains volatile organic compounds or functional treatment agents. An engagement member extends from the housing member and removably couples the housing member to a frame of the HVAC filter. An aperture formed through the body exposes a portion of the substrate member to airflow. The engagement member may comprise clips on opposing sides of the housing member. Retention tabs may extend inwardly from the sidewall to retain the substrate member. The housing member may comprise a reusable housing configured to receive replacement substrate members in various formats including absorbent pads, gel-impregnated substrates, bead-containing substrates, and powder-infused substrates. The substrate member may retain odor neutralizing compounds, antimicrobial agents, or allergen reduction compounds.

### CROSS REFERENCE TO RELATED APPLICATIONS

This application claims priority to U.S. Provisional Application No. 63/746,710, filed Jan. 17, 2025, the entire contents of which are incorporated herein by reference.

### TECHNICAL FIELD

The present disclosure pertains broadly to systems and methods for integrating scent delivery and functional air treatment into heating, ventilation, and air conditioning (HVAC) systems.

### BACKGROUND

The present disclosure relates generally to scent delivery and functional air treatment systems. More particularly, the present disclosure relates to scent delivery and functional air treatment systems configured for attachment to HVAC system components.

### SUMMARY

In one aspect, the present disclosure provides a scent delivery system for use with an HVAC filter. The scent delivery system comprises a housing member comprising a body and a sidewall extending from the body, the sidewall defining a cavity. A substrate member is disposed within the cavity, the substrate member configured to retain volatile organic compounds. An engagement member extends from the housing member, the engagement member configured to removably couple the housing member to a filter frame of the HVAC filter. An aperture is formed through the body, the aperture exposing a portion of the substrate member to airflow.

In some embodiments, the engagement member comprises a first engagement member and a second engagement member disposed on opposing sides of the housing member. Each of the first engagement member and the second engagement member may comprise a clip extending from an edge of the sidewall and configured to engage the frame of the HVAC filter.

In certain embodiments, the scent delivery system further comprises a plurality of retention tabs extending inwardly from the sidewall, the retention tabs configured to retain the substrate member within the cavity. The plurality of retention tabs may comprise four retention tabs disposed at corners of the cavity.

In various embodiments, the aperture comprises a plurality of peripheral apertures and a central aperture. The plurality of peripheral apertures may comprise four peripheral apertures disposed along edges of the body. The central aperture may comprise a decorative aperture.

In some embodiments, the substrate member comprises an absorbent material. The sidewall may extend peripherally around the body. The engagement member may extend from the sidewall. In various embodiments, the housing member comprises a reusable housing configured to receive replacement substrate members. The substrate member may comprise an absorbent pad, a gel-impregnated substrate, a bead-containing substrate, or a powder-infused substrate. In certain embodiments, the substrate member is configured to retain functional treatment agents including odor neutralizing compounds, antimicrobial agents, or allergen reduction compounds in addition to or in lieu of volatile organic compounds.

In another aspect, the present disclosure provides a scent delivery cartridge for attachment to an HVAC filter frame. The scent delivery cartridge comprises a housing member comprising a body and a sidewall extending peripherally from the body, the sidewall defining a cavity configured to receive a substrate member. A plurality of retention tabs extend inwardly from the sidewall over a portion of the cavity, the retention tabs configured to retain the substrate member within the cavity. An aperture is formed through the body. Two engagement members extend from the housing member, each engagement member comprising a clip configured to engage the HVAC filter frame.

In a further aspect, the present disclosure provides an HVAC filter assembly comprising an HVAC filter having a filter frame and a scent delivery system removably coupled to the filter frame. The scent delivery system comprises a housing member comprising a body and a sidewall extending from the body, the sidewall defining a cavity. A substrate member is disposed within the cavity, the substrate member configured to retain volatile organic compounds. An engagement member extends from the housing member and engages the filter frame. An aperture is formed through the body, the aperture exposing a portion of the substrate member.

### BRIEF DESCRIPTION OF THE DRAWINGS

FIG. 1 is a perspective view of a scent delivery system according to an embodiment of the present disclosure.

FIG. 2A is a perspective view of the scent delivery system of FIG. 1 installed on an HVAC filter.

FIG. 2B is an end view of the scent delivery system of FIG. 1 showing the first engagement member engaged with the filter frame of the HVAC filter.

FIG. 3 is a rear perspective view of the scent delivery system of FIG. 1.

FIG. 4 is a front perspective view of the scent delivery system of FIG. 1.

FIG. 5 is a perspective view of a scent delivery system according to another embodiment of the present disclosure.

FIG. 6 is a perspective view of a register assembly according to an embodiment of the present disclosure.

FIG. 7 is another perspective view of the register assembly of FIG. 6.

FIG. 8 is a perspective view of a scent delivery cartridge magnetically associated with a register.

### DETAILED DESCRIPTION

The present disclosure describes modular scent delivery and functional air treatment systems designed to integrate with existing HVAC infrastructure. The systems comprise specially configured housings containing volatile organic compound-bearing substrates or functional treatment agent-bearing substrates, with designs optimized for various mounting locations throughout an HVAC system. Multiple embodiments enable strategic placement from terminal registers to air handlers, allowing optimization of scent delivery or functional air treatment based on specific building requirements and HVAC configurations. The housings are configured as reusable housings that permit substrate members to be removed and replaced when depleted, reducing waste and operational costs compared to single-use systems.

A key aspect of the disclosed technology includes engineered mounting systems that enable secure attachment to HVAC components without system modification or performance degradation. The mounting systems accommodate various installation locations while maintaining proper orientation for optimal scent or functional agent dispersion. Integrated barrier systems preserve volatile compounds and functional treatment agents during storage and transport, while engineered substrate configurations ensure controlled release during operation.

The disclosure further describes specialized register assemblies incorporating dedicated receptacles for scent delivery cartridges or functional air treatment cartridges. These assemblies integrate air flow management features that enhance scent or functional agent distribution while maintaining proper system performance. The register assemblies enable convenient cartridge replacement and substrate refilling while providing aesthetic integration with existing HVAC infrastructure.

Through strategic placement options and engineered air flow management, the disclosed systems maximize scent or functional agent distribution effectiveness while minimizing impact on HVAC system performance. The modular nature of the system enables scalable deployment from single rooms to entire buildings, while standardized cartridge designs simplify maintenance and replacement operations. The reusable housing configurations permit substrate refilling or replacement without disposing of the entire cartridge assembly, reducing environmental impact and operational costs.

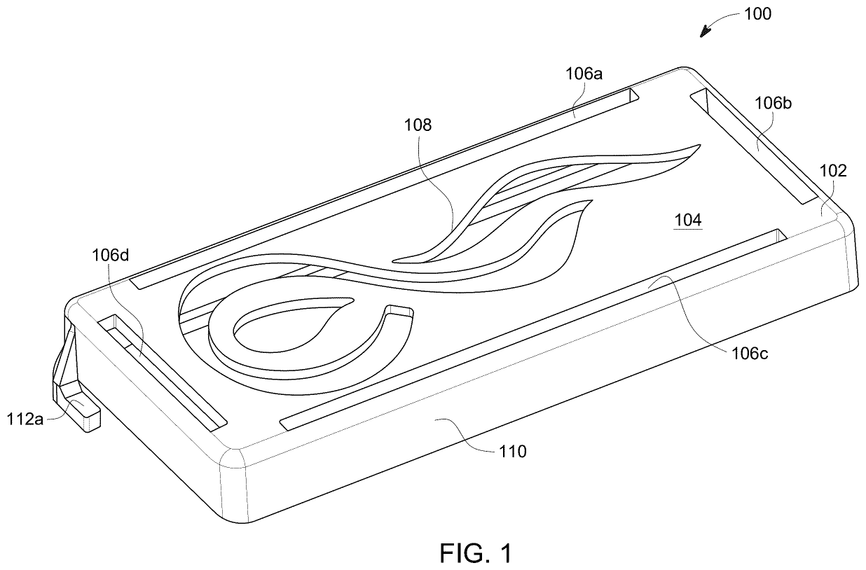

Referring to FIGS. 1 through 4, a scent delivery system 100 according to an embodiment of the present disclosure is illustrated. The scent delivery system 100 includes a housing member 102 comprising a body 104 and a sidewall 110 extending from the body 104. The sidewall 110 defines a cavity 116 configured to receive a substrate member 114. The body 104 includes a decorative aperture 108 and a plurality of peripheral apertures including a first peripheral aperture 106A, a second peripheral aperture 106B, a third peripheral aperture 106C, and a fourth peripheral aperture 106D. The scent delivery system 100 further includes a first engagement member 112A and a second engagement member 112B disposed on opposing sides of the housing member 102. Each engagement member includes a return portion 120 configured to engage a filter frame. A plurality of retention tabs including a first retention tab 118A, a second retention tab 118B, a third retention tab 118C, and a fourth retention tab 118D extend inwardly from the sidewall 110 to retain the substrate member 114 within the cavity 116.

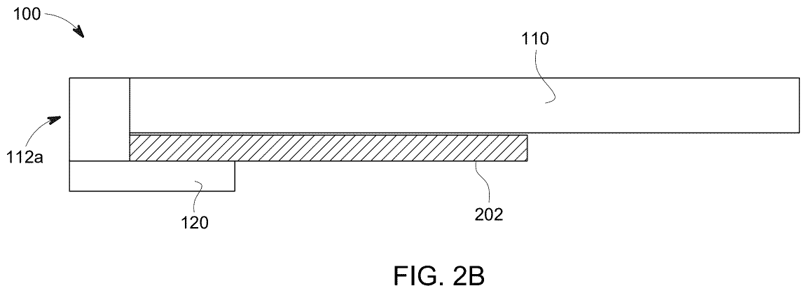

Referring to FIG. 1, the scent delivery system 100 is shown in a perspective view. The housing member 102 includes a body 104 having a planar surface and sidewall 110. The sidewall 110 extends peripherally around the body 104 to define the cavity 116. The body 104 includes a decorative aperture 108 formed therethrough, as well as a plurality of peripheral apertures including a first peripheral aperture 106A, a second peripheral aperture 106B, a third peripheral aperture 106C, and a fourth peripheral aperture 106D. The decorative aperture 108 and the peripheral apertures 106A, 106B, 106C, 106D expose the substrate member 114 (see FIG. 3) to airflow when the scent delivery system 100 is installed on an HVAC filter.

The housing member 102 further includes a first engagement member 112A extending from the housing member 102. As shown in FIG. 1, the first engagement member 112A is disposed at one side of the housing member 102 and is configured to removably couple the housing member 102 to a frame of an HVAC filter. The first engagement member 112A comprises a clip that can engage the filter frame to secure the scent delivery system 100 in position on the HVAC filter. A second engagement member is provided on the opposing side of the body 104 and is shown and described in other figures.

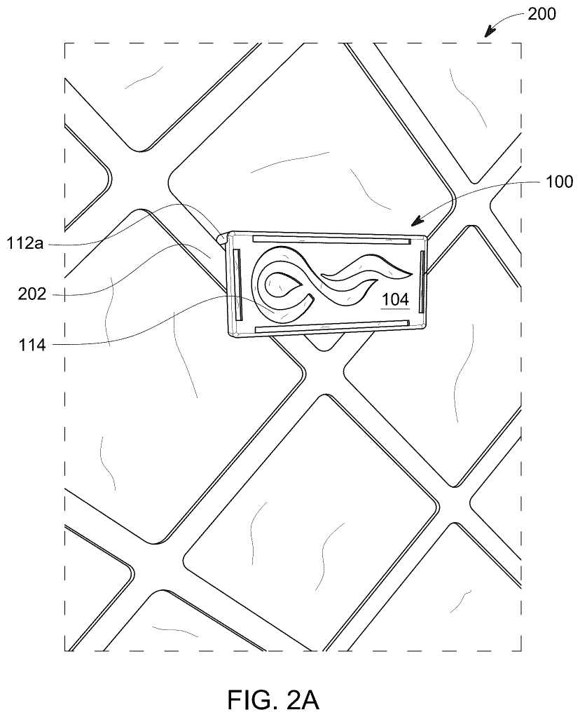

Referring to FIG. 2A, the scent delivery system 100 is shown installed on an HVAC filter 200. The HVAC filter 200 includes a filter frame 202 that surrounds filter media. The scent delivery system 100 is removably coupled to the filter frame 202 via the first engagement member 112A. The body 104 of the housing member 102 is visible, with the substrate member 114 exposed through the decorative aperture 108 and the peripheral apertures. When air flows through the HVAC filter 200, the airflow passes over the substrate member 114, causing volatile organic compounds retained by the substrate member 114 to be dispersed into the air stream.

The filter frame 202 may comprise intersecting support members that define a V-shaped channel at their intersection. The engagement members 112A, 112B are configured to engage different portions of the filter frame 202, including the V-shaped channel formed at the intersection of support members. The return portion 120 of each engagement member is sized to fit within the V-shaped channel, permitting the scent delivery system 100 to be positioned at various locations along the filter frame 202.

Referring to FIG. 2B, an end view of the scent delivery system 100 is shown with the first engagement member 112A engaged with the filter frame 202. Each of the first engagement member 112A and the second engagement member 112B (see FIG. 3) extends from an edge of the sidewall 110 or the alternatively the body 104. Each engagement member includes a return portion 120 that extends away from the sidewall 110 and returns toward the housing member 102. The return portion 120 is configured to engage a rear surface of the filter frame 202, while a rear surface of the sidewall 110 is configured to engage a front surface of the filter frame 202. The filter frame 202 is captured between the return portion 120 of the engagement member and the rear surface of the sidewall 110.

The return portion 120 of each engagement member defines a gap between the return portion 120 and the rear surface of the sidewall 110. The gap is sized to receive the filter frame 202 therethrough. In some embodiments, the gap corresponds to a thickness of the filter frame 202, such that the engagement member frictionally engages the filter frame 202 when the scent delivery system 100 is installed on the HVAC filter 200.

The housing member 102 is formed from a resilient material, such as a thermoplastic polymer, that permits elastic deformation of the first engagement member 112A and the second engagement member 112B during attachment to and removal from the filter frame 202. When the scent delivery system 100 is positioned adjacent the filter frame 202, the return portion 120 deflects to permit the filter frame 202 to enter the gap. Upon full insertion of the filter frame 202 into the gap, the return portion 120 springs back to capture the filter frame 202 between the return portion 120 and the rear surface of the sidewall 110. This snap-fit engagement permits tool-free installation and removal of the scent delivery system 100.

Referring to FIG. 3, a rear perspective view of the scent delivery system 100 is shown. The cavity 116 is visible, with the substrate member 114 disposed within the cavity 116. The substrate member 114 comprises an absorbent material configured to retain volatile organic compounds or functional treatment agents. In some embodiments, the substrate member 114 may comprise a foam pad, a fibrous material, a felt pad, a cellulose pad, or other absorbent substrate capable of holding and releasing scent compounds or functional treatment agents. In various embodiments, the substrate member 114 is configured to receive and retain liquid formulations, gel formulations, solid beads, granular materials, or powder formulations containing volatile organic compounds or functional treatment agents.

As shown in FIG. 3, the scent delivery system 100 includes both a first engagement member 112A and a second engagement member 112B. The first engagement member 112A and the second engagement member 112B are disposed on opposing sides of the housing member 102. Each of the first engagement member 112A and the second engagement member 112B comprises a clip extending from an edge of the sidewall 110 and configured to engage the filter frame 202 (see FIG. 2A) of the HVAC filter 200.

The first engagement member 112A and the second engagement member 112B are disposed at diagonally opposing corners of the housing member 102. This diagonal arrangement permits single-handed installation of the scent delivery system 100 on the filter frame 202 by engaging the first engagement member 112A with a first side of the filter frame 202 and subsequently pivoting the housing member 102 to engage the second engagement member 112B with a second side of the filter frame 202.

The first engagement member 112A and the second engagement member 112B are oriented such that the respective return portions 120 extend in opposite directions relative to the housing member 102. This opposing orientation permits the engagement members 112A, 112B to engage opposite sides of the filter frame 202, securing the scent delivery system 100 to the HVAC filter 200 against displacement.

The scent delivery system 100 further comprises a plurality of retention tabs configured to retain the substrate member 114 within the cavity 116. As shown in FIG. 3, the retention tabs include a first retention tab 118A, a second retention tab 118B, a third retention tab 118C, and a fourth retention tab 118D. The retention tabs 118A, 118B, 118C, 118D extend inwardly from the sidewall 110 over a portion of the cavity 116. The four retention tabs 118A, 118B, 118C, 118D are disposed at corners of the cavity 116. The retention tabs 118A, 118B, 118C, 118D engage the substrate member 114 to prevent the substrate member 114 from dislodging from the cavity 116 during handling or operation.

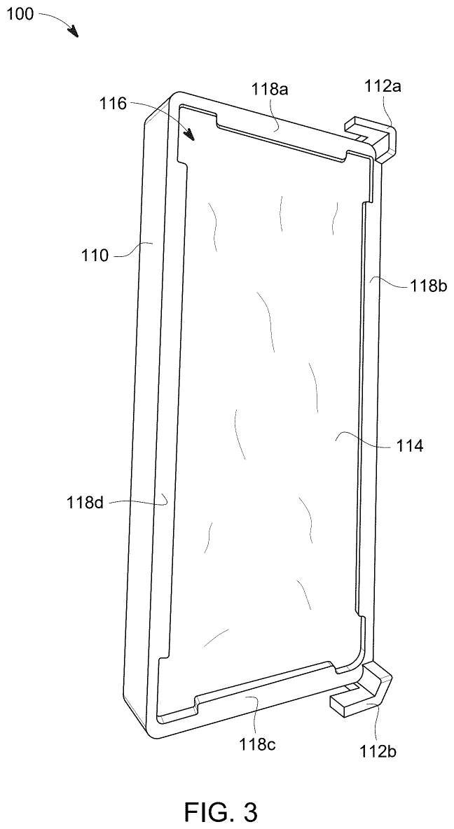

Referring to FIG. 4, a front perspective view of the scent delivery system 100 is shown. The body 104 is visible with the decorative aperture 108 and the peripheral apertures 106A, 106B, 106C, 106D formed therethrough. The substrate member 114 is visible through the decorative aperture 108 and the peripheral apertures 106A, 106B, 106C, 106D. The sidewall 110 extends around the periphery of the body 104. The peripheral apertures 106A, 106B, 106C, 106D are disposed along edges of the body 104, while the decorative aperture 108 is centrally located on the body 104.

The scent delivery system 100 accommodates substrate members in various refill formats. In some embodiments, the substrate member 114 comprises an absorbent pad pre-impregnated with volatile organic compounds or functional treatment agents. In other embodiments, the substrate member 114 comprises an unloaded absorbent pad configured to receive liquid scent formulations or liquid functional treatment formulations applied by the user. In further embodiments, the substrate member 114 comprises a gel-impregnated substrate configured to release volatile organic compounds or functional treatment agents at a controlled rate. In still other embodiments, the substrate member 114 comprises a container holding scented beads, odor-neutralizing beads, antimicrobial beads, or allergen-reducing beads, wherein the container includes apertures permitting airflow communication with the beads. In yet further embodiments, the substrate member 114 comprises a porous substrate infused with or coated with powder formulations containing scent compounds or functional treatment agents. The cavity 116 is dimensioned to receive substrate members in any of these formats, permitting the user to select the refill format best suited to the intended application.

In operation, a user may insert the substrate member 114 into the cavity 116 of the housing member 102. The retention tabs 118A, 118B, 118C, 118D retain the substrate member 114 in position. The user may apply volatile organic compounds to the substrate member 114, such as by applying scented oil with a dropper. The user then attaches the scent delivery system 100 to an HVAC filter 200 by engaging the first engagement member 112A and the second engagement member 112B with the filter frame 202. When the HVAC system operates, air flowing through the HVAC filter 200 passes over the substrate member 114 exposed through the apertures, dispersing the volatile organic compounds throughout the conditioned space. The housing member 102 is configured as a reusable housing that permits repeated installation and removal of substrate members. When the volatile organic compounds retained by the substrate member 114 are depleted, the user may remove the depleted substrate member 114 from the cavity 116 and insert a replacement substrate member. Alternatively, the user may recharge the substrate member 114 in situ by applying additional volatile organic compounds to the substrate member 114 while the substrate member 114 remains disposed within the cavity 116. The reusable housing member 102 accommodates multiple refill cycles without degradation of the engagement members 112A, 112B or the retention tabs 118A, 118B, 118C, 118D.

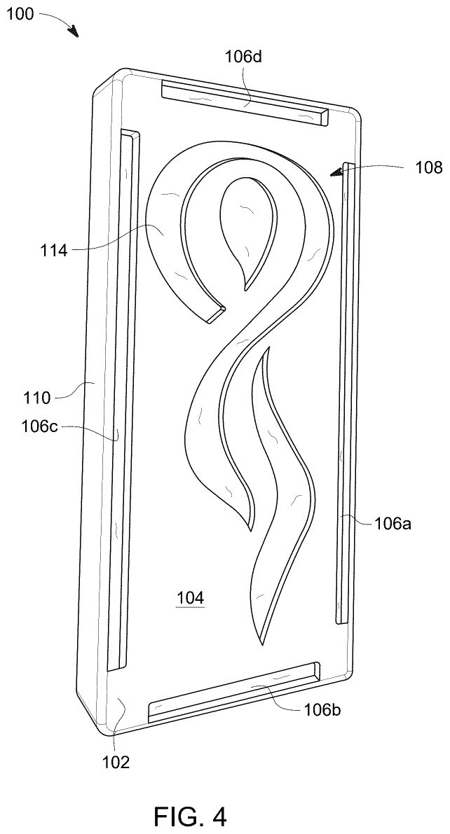

Referring to FIG. 5, a scent delivery system 500 according to another embodiment is illustrated. The scent delivery system 500 includes a housing member 502 comprising a first planar substrate 504 and a second planar substrate 506 connected at their adjoining peripheries. A scented member 508 is disposed within an internal cavity defined by the housing member 502. The scent delivery system 500 further includes a first engagement member 510 and a second engagement member 512 extending from the housing member 502. A cover 514 is applied to the first planar substrate 504.

The housing member 502 can be fabricated from any number of methods that would be known to one of ordinary skill in the art. The housing member 502 needs to possess a cavity that receives the scented member 508 and has covers that keep the scented member 508 fresh when not in use and can be removed in order to promote airflow when in a deployed state. The housing member 502 is configured as a reusable housing permitting the scented member 508 to be removed and replaced with a fresh scented member when depleted. The cavity of the housing member 502 is dimensioned to receive scented members in various formats including absorbent pads, gel packs, bead-filled pouches, and powder-infused substrates.

In some instances, the scented member 508 is covered on each side by a micro-perforated film or layer. This film not only helps retain the scented member 508, but will provide paths for the communication of fluid such as air through the scented member 508. Both front sides and back sides of the scented member 508 are provided with micro-perforated film.

In a first embodiment, the scent delivery system is configured specifically for attachment to an HVAC filter. The engagement members comprise elongated arms extending substantially perpendicular from the housing member 502. Each arm includes a terminal portion that curves inward toward the opposing arm, creating a gripping surface. The arms are formed from a resilient material allowing elastic deformation during filter engagement. The housing member may include reinforcement ribs extending along the first planar surface to maintain structural integrity. The substrate member is secured within the internal cavity through mechanical interference, adhesive bonding, or thermal bonding.

In a second embodiment, the scent delivery system is adapted for register mounting. The housing member comprises a low-profile configuration. The engagement members comprise tabbed projections dimensioned to engage standard register slot spacing. Each tabbed projection includes serrated edges to enhance mechanical engagement with register surfaces. The housing member incorporates airflow channels along the first planar surface to reduce air resistance. The substrate member is die-cut to match the airflow channel pattern, maximizing scent dispersion while minimizing pressure drop.

In a third embodiment, the scent delivery system is engineered for return air duct installation. The housing member exhibits an airfoil-shaped profile with a leading edge thickness. The engagement members comprise spring-loaded clips with rubber-coated contact surfaces to prevent duct damage. Multiple substrate layers are stacked within the internal cavity, separated by air gaps to enhance volatile organic compound release. Alignment pins extend through the substrate layers to maintain position during air flow.

In a fourth embodiment, the scent delivery system is designed for air handler unit proximity. The housing member is constructed from high-temperature thermoplastic rated for continuous exposure to 180 degrees Fahrenheit. The engagement members comprise adjustable mounting brackets allowing orientation optimization relative to air flow. The substrate member incorporates a honeycomb structure providing increased surface area for volatile organic compound retention. Internal baffles direct air flow through the honeycomb structure, ensuring maximum scent extraction. A temperature-responsive valve system modulates volatile organic compound release based on air stream temperature.

The substrate member may be constructed as a multi-layer composite incorporating microporous materials, activated carbon, or zeolites. The housing member may include visual indicators showing remaining volatile organic compound capacity. The removable barrier member may incorporate a staged release system allowing partial volatile organic compound activation. In various embodiments, the substrate member is configured to retain functional treatment agents in addition to or in lieu of scent compounds. Functional treatment agents include odor neutralizing compounds configured to chemically react with or adsorb malodorous molecules present in the airstream, antimicrobial agents configured to inhibit or destroy bacteria, viruses, mold, or fungi present in the airstream, and allergen reduction compounds configured to bind, neutralize, or deactivate common allergens including pet dander, pollen, dust mite proteins, and mold spores. The substrate member may retain combinations of scent compounds and functional treatment agents to provide both fragrance delivery and air treatment functionality. In some embodiments, the functional treatment agents comprise photocatalytic compounds activated by ambient light to decompose organic pollutants in the airstream.

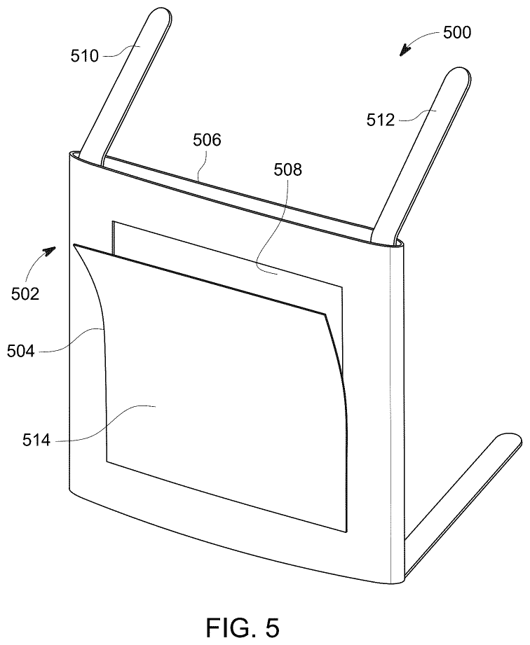

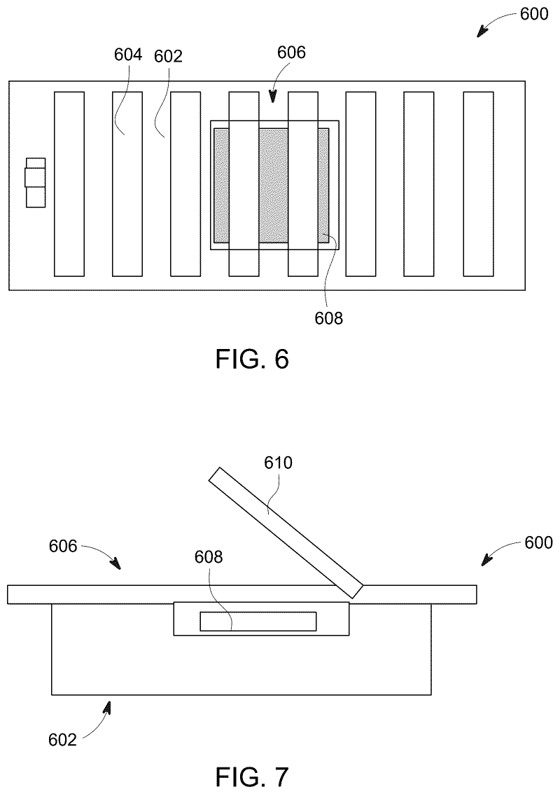

Referring to FIGS. 6 and 7, a register assembly according to an embodiment of the present disclosure is illustrated. The register assembly includes a frame member 600 defining a central air passage 602 for airflow. A plurality of louvers 604 are arranged within the frame member 600 to direct air through the central air passage 602. A hinged receptacle 606 is mounted within the central air passage 602 and is configured to house a scent delivery cartridge 608. The hinged receptacle 606 includes a cover 610 that secures the scent delivery cartridge 608 in place during operation.

The hinged receptacle 606 is mounted within the central air passage 602 and is designed to pivot between an open position and a closed position. In the open position, the scent delivery cartridge 608 can be inserted or replaced. In the closed position, the receptacle 606 aligns with the airflow through the louvers 604, allowing air to pass over the scent delivery cartridge 608. This airflow facilitates the dispersion of volatile organic compounds (VOCs) from the cartridge into the surrounding air.

The hinged receptacle 606 includes a cover 610 that secures the scent delivery cartridge 608 in place during operation, as well as a cavity that receives the scent delivery cartridge 608. The modular design of the assembly enables easy maintenance and replacement of the scent delivery cartridge 608 while maintaining the aesthetic and functional integrity of the register.

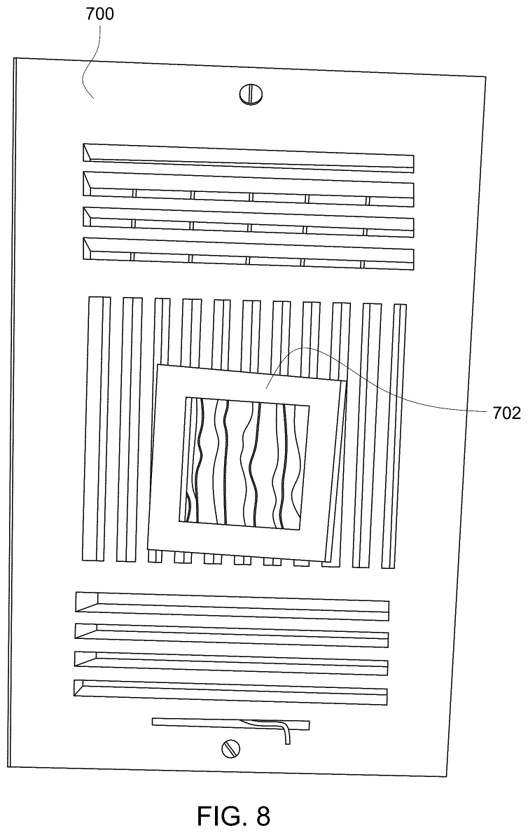

Referring to FIG. 8, a scent delivery cartridge 700 according to another embodiment is illustrated. The scent delivery cartridge 700 is configured for magnetic association with a register 702.

In some instances, the frame of the scent delivery cartridge 700 is made from a polygonal arrangement of structural material that when assembled includes a window. This window is covered on the front and back with micro-perforated layers. The scent delivery cartridge 700 can act as a pocket and a user can insert and remove a scent delivery member into the frame. The scent delivery member can be, for example, a sponge, a cloth, a nano or micro-porous material, a gel pack, a bead-filled pouch, a powder-infused substrate, and the like. The frame of the scent delivery cartridge 700 is configured as a reusable housing that permits repeated insertion and removal of scent delivery members, allowing the user to refill or replace the scent delivery member when depleted without replacing the entire cartridge. The scent delivery member may contain scent compounds, odor neutralizing agents, antimicrobial agents, allergen reduction compounds, or combinations thereof.

The corresponding structures, materials, acts, and equivalents of all means or step plus function elements in the claims below are intended to include any structure, material, or act for performing the function in combination with other claimed elements as specifically claimed. The description of the present technology has been presented for purposes of illustration and description, but is not intended to be exhaustive or limited to the present technology in the form disclosed. Many modifications and variations will be apparent to those of ordinary skill in the art without departing from the scope and spirit of the present technology. Exemplary embodiments were chosen and described in order to best explain the principles of the present technology and its practical application, and to enable others of ordinary skill in the art to understand the present technology for various embodiments with various modifications as are suited to the particular use contemplated.

If any disclosures are incorporated herein by reference and such incorporated disclosures conflict in part and/or in whole with the present disclosure, then to the extent of conflict, and/or broader disclosure, and/or broader definition of terms, the present disclosure controls. If such incorporated disclosures conflict in part and/or in whole with one another, then to the extent of conflict, the later-dated disclosure controls.

The terminology used herein can imply direct or indirect, full or partial, temporary or permanent, immediate or delayed, synchronous or asynchronous, action or inaction. For example, when an element is referred to as being “on,” “connected” or “coupled” to another element, then the element can be directly on, connected or coupled to the other element and/or intervening elements may be present, including indirect and/or direct variants. In contrast, when an element is referred to as being “directly connected” or “directly coupled” to another element, there are no intervening elements present.

The terminology used herein is for the purpose of describing particular embodiments only and is not intended to be necessarily limiting of the disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence or addition of one or more other features, integers, steps, operations, elements, components, and/or groups thereof.

Example embodiments of the present disclosure are described herein with reference to illustrations of idealized embodiments (and intermediate structures) of the present disclosure. As such, variations from the shapes of the illustrations as a result, for example, of manufacturing techniques and/or tolerances, are to be expected. Thus, the example embodiments of the present disclosure should not be construed as necessarily limited to the particular shapes of regions illustrated herein, but are to include deviations in shapes that result, for example, from manufacturing.

In this description, for purposes of explanation and not limitation, specific details are set forth, such as particular embodiments, procedures, techniques, etc. in order to provide a thorough understanding of the present disclosure. However, it will be apparent to one skilled in the art that the present disclosure may be practiced in other embodiments that depart from these specific details.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” or “according to one embodiment” (or other phrases having similar import) at various places throughout this specification are not necessarily all referring to the same embodiment. Furthermore, the particular features, structures, or characteristics may be combined in any suitable manner in one or more embodiments. Furthermore, depending on the context of discussion herein, a singular term may include its plural forms and a plural term may include its singular form. Similarly, a hyphenated term (e.g., “on-demand”) may be occasionally interchangeably used with its non-hyphenated version (e.g., “on demand”), a capitalized entry (e.g., “Software”) may be interchangeably used with its non-capitalized version (e.g., “software”), a plural term may be indicated with or without an apostrophe (e.g., PE's or PEs), and an italicized term (e.g., “N+1”) may be interchangeably used with its non-italicized version (e.g., “N+1”). Such occasional interchangeable uses shall not be considered inconsistent with each other.

Also, some embodiments may be described in terms of “means for” performing a task or set of tasks. It will be understood that a “means for” may be expressed herein in terms of a structure, such as a processor, a memory, an I/O device such as a camera, or combinations thereof. Alternatively, the “means for” may include an algorithm that is descriptive of a function or method step, while in yet other embodiments the “means for” is expressed in terms of a mathematical formula, prose, or as a flow chart or signal diagram.

What is claimed is:

1. A scent delivery system for use with an HVAC filter, comprising:
a housing member comprising a body and a sidewall extending from the body, the sidewall defining a cavity;
a substrate member disposed within the cavity, the substrate member configured to retain volatile organic compounds;
an engagement member extending from the housing member, the engagement member configured to removably couple the housing member to a filter frame of the HVAC filter; and
an aperture formed through the body, the aperture exposing a portion of the substrate member to airflow.

2. The scent delivery system of claim 1, wherein the engagement member comprises a first engagement member and a second engagement member disposed on opposing sides of the housing member.

3. The scent delivery system of claim 2, wherein each of the first engagement member and the second engagement member comprises a clip extending from an edge of the sidewall, the clip including a return portion configured to engage a rear surface of the filter frame while a rear surface of the sidewall engages a front surface of the filter frame.

4. The scent delivery system of claim 1, further comprising a plurality of retention tabs extending inwardly from the sidewall, the retention tabs configured to retain the substrate member within the cavity.

5. The scent delivery system of claim 4, wherein the plurality of retention tabs comprises four retention tabs disposed at corners of the cavity.

6. The scent delivery system of claim 1, wherein the aperture comprises a plurality of peripheral apertures and a central aperture.

7. The scent delivery system of claim 6, wherein the plurality of peripheral apertures comprises four peripheral apertures disposed along edges of the body.

8. The scent delivery system of claim 1, wherein the substrate member comprises an absorbent material.

9. The scent delivery system of claim 6, wherein the central aperture comprises a decorative aperture.

10. The scent delivery system of claim 1, wherein the sidewall extends peripherally around the body.

11. The scent delivery system of claim 1, wherein the engagement member extends from the sidewall.

12. A scent delivery cartridge for attachment to an HVAC filter frame, comprising:
a housing member comprising a body and a sidewall extending peripherally from the body, the sidewall defining a cavity configured to receive a substrate member;
a plurality of retention tabs extending inwardly from the sidewall over a portion of the cavity, the retention tabs configured to retain the substrate member within the cavity;
an aperture formed through the body; and
two engagement members extending from the housing member, each engagement member comprising a clip including a return portion configured to engage a rear surface of the HVAC filter frame while a rear surface of the sidewall engages a front surface of the HVAC filter frame.

13. The scent delivery cartridge of claim 12, wherein the plurality of retention tabs comprises a first retention tab, a second retention tab, a third retention tab, and a fourth retention tab.

14. The scent delivery cartridge of claim 12, wherein the aperture comprises a decorative aperture and a plurality of peripheral apertures.

15. The scent delivery cartridge of claim 12, further comprising a substrate member disposed within the cavity and retained by the plurality of retention tabs.

16. The scent delivery cartridge of claim 15, wherein the substrate member comprises an absorbent material configured to retain volatile organic compounds.

17. An HVAC filter assembly comprising:
an HVAC filter having a filter frame;
a scent delivery system removably coupled to the filter frame, the scent delivery system comprising:
a housing member comprising a body and a sidewall extending from the body, the sidewall defining a cavity;
a substrate member disposed within the cavity, the substrate member configured to retain volatile organic compounds;
an engagement member extending from the housing member and engaging the filter frame; and
an aperture formed through the body, the aperture exposing a portion of the substrate member.

18. The HVAC filter assembly of claim 17, wherein the engagement member comprises a first engagement member and a second engagement member, each of the first engagement member and the second engagement member comprising a clip including a return portion engaging a rear surface of the filter frame while a rear surface of the sidewall engages a front surface of the filter frame.

19. The HVAC filter assembly of claim 17, wherein the scent delivery system further comprises a plurality of retention tabs extending inwardly from the sidewall and retaining the substrate member within the cavity.

20. The HVAC filter assembly of claim 17, wherein the aperture comprises a decorative aperture and a plurality of peripheral apertures formed through the body.
